(19) United States
(12) Patent Application Publication
(10) Pub. No.: US 2022/0079526 A1
(43) Pub. Date: Mar. 17, 2022

(54) APPARATUS AND METHOD FOR TRACKING HEAD MOTION IN MAGNETIC RESONANCE IMAGING (MRI)

(71) Applicant: KONINKLIJKE PHILIPS N.V., ElNDHOVEn (NL)

(72) Inventor: Olli Tapio Friman, GAINESVILLE, FL (US)

(21) Appl. No.: 17/419,332

(22) PCT Filed: Dec. 23, 2019
(86) PCT No.: PCT/EP2019/086891

§ 371 Date: Jun. 29, 2021

Related U.S. Application Data
(60) Provisional application No. 62/787,859, filed on Jan. 3, 2019.

(51) Int. Cl. A61B 5/00; A61B 5/11; A61B 5/055

(52) U.S. Cl. CPC A61B 5/70; A61B 5/1114; A61B 6/032; A61B 5/721; A61B 5/055

(57) ABSTRACT

A headrest (10) for an imaging device (24) includes a base (12); a head cradle (14) having a pivot connection (16) or rolling connection (18) with the base; and a sensor (22) configured to measure a pivot angle (θ) of the head cradle about a pivot axis (A) of the pivot connection of the head cradle with the base or a roll position (P) of the rolling connection of the head cradle with the base.

### FIELD

The following relates generally to the imaging arts and more particularly to the brain imaging arts, the magnetic resonance imaging (MRI) arts, head motion tracking and motion compensation arts, and to related arts.

### BACKGROUND

Medical imaging devices include very complex systems such as magnetic resonance imaging (MRI) devices, transmission computed tomography (CT) imaging devices, emission imaging systems such as positron emission tomography (PET) imaging devices and gamma cameras for single photon emission computed tomography (SPECT) imaging, hybrid systems that provide multiple modalities in a single device, e.g. a PET/CT or SPECT/CT imaging device, and imaging devices designed for guiding biopsies or other interventional medical procedures, commonly referred to as image guided therapy (iGT) devices. These are merely illustrative examples. Medical imaging of the head, and most often the brain, finds a wide range of clinical applications such as assessing traumatic head injury, identifying and monitoring brain tumors, performing functional MM imaging (fMRI) to directly image areas of neurological activity, and so forth.

The following discloses a new and improved systems and methods.

### SUMMARY

In one disclosed aspect, a headrest for an imaging device includes a base; a head cradle having a pivot connection or rolling connection with the base; and a sensor configured to measure a pivot angle of the head cradle about a pivot axis of the pivot connection of the head cradle with the base or a roll position of the rolling connection of the head cradle with the base.

In another disclosed aspect, a method of measuring a motion shift of a head resting in a head cradle having a pivot connection or rolling connection with a base is disclosed. The method includes: using a sensor, measuring a pivot angle of the head cradle about a pivot axis of the pivot connection of the head cradle with the base or a roll position of the rolling connection of the head cradle with the base; using an imaging device, acquiring an image of the head resting in the head cradle; and with at least one electronic processor, computing motion shifts of voxels of the image of the head resting in the head cradle due to motion of the head using the measured pivot angle or roll position.

One advantage resides in providing a head rest for use in an imaging procedure which facilitates accurate tracking of head motion.

Another advantage resides in providing a head rest for use in an imaging procedure to prevent or reduce undesired and/or difficult to measure sliding between the skin and the skull.

Another advantage resides in providing a head rest for use in an imaging procedure with a sensor that provides information on head motion that is useful (alone or in combination with imaging data) for accurately assessing head position.

Another advantage resides in providing a head rest for correcting for patient head movement in Mill imaging data after image acquisition to determine a better position for the head during imaging.

Another advantage resides in providing a head rest for use in an imaging procedure that improves patient comfort and does not require additional setup or configuration work for an Mill technician.

Another advantage resides in providing one or more of the foregoing benefits using a head rest, in which the tracked head motion is not dependent on a size of the head.

Another advantage resides in providing one or more of the foregoing benefits using a head rest, in which the tracked head motion is not dependent on a shape of the head.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a first embodiment of a head rest for an imaging device according to one aspect.

FIG. 2 diagrammatically illustrates a second embodiment of a head rest for an imaging device according to another aspect.

FIG. 3 shows an exemplary flow chart operation of a system of FIG. 1.

FIGS. 4 and 5 show motion calculations of the head rest of FIGS. 1 and 2, respectively.

### DETAILED DESCRIPTION

Existing head imaging equipment typically uses some form of head stabilization to reduce head motion of a patient during imaging. However, it is recognized herein that restraining head motion by holding the skin of the head in place does not provide sufficient restriction to restrict head movement. This is because the restraint contacts the skin covering the skull, but the skull remains free to move by some amount within the skin covering. In brain surgical procedures, the head is held in place by ‘spikes’ that penetrate the skin and are attached to the skull. This solution is used for brain surgery due to the requirement to strictly immobilize the brain during surgery; however, it is generally impractical for brain imaging procedures. For routine MRI, PET, or other brain imaging procedures, motion tracking is typically performed so that motion can be compensated for, instead of totally restricted. It has been found that the most significant movement is the rotation of the head in the axial plane (i.e., rotating side to side).

The problem with restriction of head movement in this case is that the skin/skull contact is extremely well ‘lubricated’ (i.e., the skull moves relatively freely against the skin). The difference between rolling one's head on the surface versus rotating one's head by letting the skin on the back of the head slide on the skull is very small, yet this difference is important for motion tracking during brain imaging procedures. This problem can persist even in camera tracking based on facial feature recognition, since the camera images the skin and not the skull which may be moving within the skin.

Head motion of a patient in a supine position (e.g., lying on his/her back and facing up) can be one of two distinct types of side-to-side rotating head motions: movement of the head as a whole; and movement of the skull relative to the skin (e.g., the skin remains in place on a headrest, but the skull moves within the skin). Of these two mechanisms, it is recognized herein that it is much easier to correct for the whole-head movement.

Based on the foregoing insights, the following discloses improved headrests that constrain the head motion to whole-head movement. The head is positioned in a wedge-shaped or other receiving cradle that holds the head firmly, combined with a rolling support or pivot mount of the head cradle on a head coil or other underlying support (generally referred to herein as the “base” of the headrest). This provides a well-defined geometry of side-to-side rotating head motion. Additionally, the pivot angle or roll position of the headrest is measured by a suitable sensor (e.g., an inclinometer, laser-based optical sensor, or a rotational encoder in the case of a pivot mount, or so forth) and this measurement serves as an additional input for performing motion correction of the imaging data of the head.

With this additional pivot angle or roll position input, and a priori knowledge of the location of the surface of the base on which the cradle rolls, or of the pivot axis in the case of a pivot connection, in the MRI frame of reference (this a priori knowledge is known from the position of the headrest on the patient support whose position is known in the MM frame of reference), a purely geometric formula can be used to compute the shift of each voxel of the head in the MRI frame of reference due to the head motion. Advantageously, the same geometric formula applies regardless of the size or detailed shape of the head, as the per-voxel shift depends only upon its geometric position respective to the pivot axis or rolling surface.

While disclosed in the illustrative examples for magnetic resonance (MR), the approach could be applied in computed tomography (CT) imaging, positron emission tomography (PET) imaging, or any other medical imaging technique in which head motion is permitted but should be accurately tracked. The illustrative examples are directed to side-to-side rotational motion in the axial plane, but analogous approach could be used for nodding rotational motion in the sagittal plane.

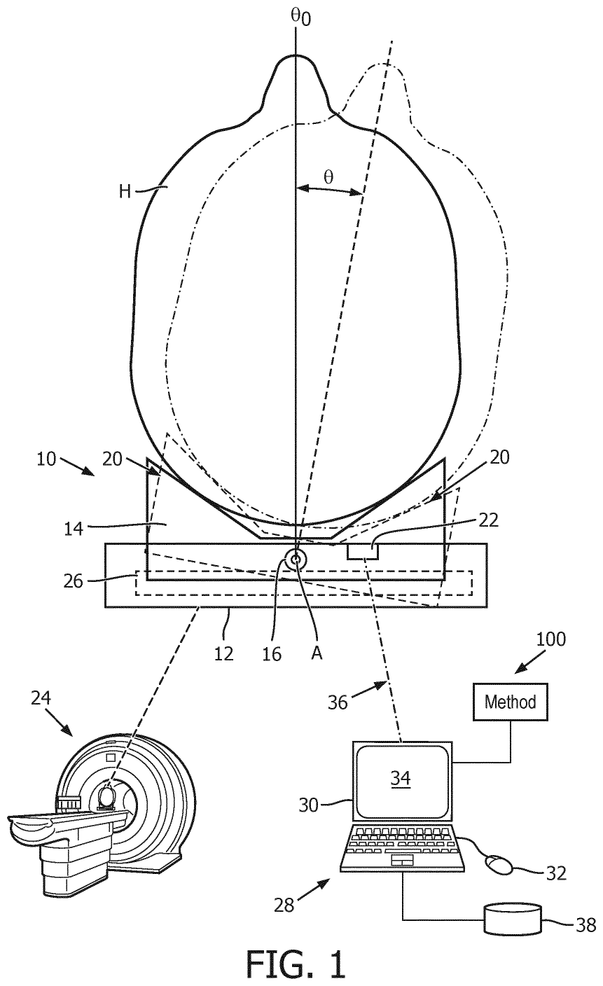

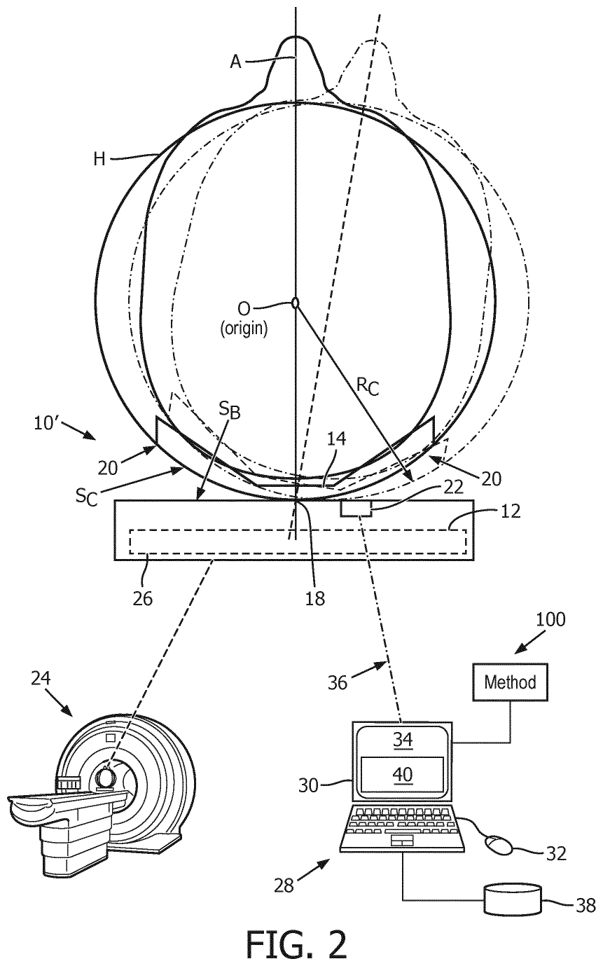

With reference to FIG. 1, an exemplary headrest 10 is illustrated. As shown in FIG. 1, the headrest 10 includes a base 12 and a head cradle 14. The head cradle 14 is connected to the base 12 with a pivot connection 16 (or, in another embodiment, by a rolling connection 18 as shown in FIG. 2). The illustrative head cradle 14 includes wedge-shaped portions 20 disposed at opposing ends of the head cradle to receive a head H of a patient to be imaged. More generally, the head cradle 14 is shaped with a recess, depression, or other structure for receiving and holding the head H in the head cradle 14. The cradle 14 can be made from any suitable material (e.g., plastic). The base 12 is stationary during the imaging, and can be variously embodied. For example, the base 12 may be a box, disk, or other structure that is optionally secured with the patient couch or other patient support, e.g. by designated fasteners at a specific location on the patient support platen or plate. In some embodiments, the base 12 may actually be the patient couch which in these embodiments has the pivot connection 16 integrally built in, or may be a sliding patient transport plate that moves between the patient loading couch and the bore of the MRI and (in these embodiments) has the pivot connection 16 integrally built in. As further examples, in some embodiments, which are particularly suitable for MRI, the base 12 may be an MR head coil designed for placement behind (i.e. underneath) the head of the supine patient, and which has the pivot connection 16. These are merely illustrative examples.

A sensor 22 is disposed in or on or with the base 12 (e.g., embedded within the base, attached to the pivot connection 16, attached to a surface of the base, and so forth) or positioned proximate to the base (e.g. on the patient couch or patient support platen or plate. The sensor 22 can include an inclinometer, a laser-based optical sensor, or a rotational encoder (or any other suitable sensor). The sensor 22 is configured to measure a pivot angle θ of the head cradle 14 about a pivot axis A of the pivot connection 16. The pivot angle θ is suitably measured respective to a reference angle θ₀. FIG. 1 shows the head H and cradle 14 positioned at reference angle θ₀ using solid lines; and at a positive angle indicated as angle θ using dash-dot lines.

The headrest 10 is configured for use with an imaging device 24 configured to obtain one or more images of the patient's head disposed in the head cradle 14. FIG. 1 shows an MRI device 24, but the headrest 10 can be used for any other suitable imaging device (e.g., a CT imaging device, PET imaging device, combined CT/PET scanner, or so forth). As shown in FIG. 1, in the case of MRI an MR head coil 26 can optionally be disposed on or in the base 12 (as diagrammatically shown) and/or in the head cradle 14. The MR head coil 26 may be a single coil or may be a coil array, e.g. to support parallel MR head imaging. Placing the MR head coil 26 on or in the base 12, as shown, simplifies porting the received MR signal off the coil 26 since the base 12 is stationary during imaging; on the other hand, placing the MR head coil on or in the head cradle 14 places it in closer proximity to the head, but may require more complex wiring to port the MR signal off the pivoting head cradle.

The sensor 22 is in communication (e.g., operatively connected) with a workstation 28 comprising a computer or other electronic data processing device with at least one electronic processor 30, and optionally including other typical components such as at least one user input device (e.g., a mouse, a keyboard, a trackball, and/or the like) 32, and a display device 34. It should be noted that these components can be variously distributed. In another contemplated approach, the electronic processor 30 is embodied at least partly as a cloud computing resource or other remote server computer(s). The sensor 22 may have a wired connection or may communicate via a wireless link 36, such as a Bluetooth link, Wi-Fi link, and/or the like. The electronic processor 30 also optionally includes or has access to one or more databases or non-transitory storage media 38. The non-transitory storage media 34 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth. The display device 34 is configured to display MRI images, and optionally may provide a graphical user interface (GUI) including one or more fields to receive a user input from the user input device 32, e.g. to configure an MRI scan performed by the MM imaging device 24 under control of the computer 28.

The processor 30 is programmed to reconstruct an MRI image of the head H from magnetic resonance data acquired by the MRI scanner 24 (or, in other embodiments, to reconstruct a PET image reconstructed from PET data acquired by a PET scanner, or so forth for other imaging modalities). The processor 30 is further configured to compute shifts of voxels of a reconstructed image of the head H resting in the head cradle 14 respective to the reference position of the head defined by a reference pivot angle θ₀ (or roll position, in the case of the embodiment of FIG. 2 described elsewhere) of the head cradle 14 using the pivot angle θ or (roll position) measured by the sensor 22. In some examples, the processor 30 is programmed to compute shifts of voxels of the image of the head resting in the head cradle 14, and compensate for positions of the voxels based on a measured roll position θ(t) of the head cradle from the received roll position measurement and measured coordinates of the voxels. To do so, the processor 30 is programmed to receive the pivot angle measurement of the pivot angle θ of the head cradle 14 from the sensor 22, along with one or more images of the head resting in the head cradle from the imaging device 24. It should be noted that by action of the cradle 14 which holds the head H in a recess or the like, and by further action of the pivot connection 16, side-to-side movement of the skull within the skin is unlikely. Rather, the cradle 14 and pivot connection 16 operate to support whole-head movement in which side-to-side movement of the head occurs by way of pivoting of the cradle 14 (and the whole head H in the cradle) about the pivot axis A. This is diagrammatically shown in FIG. 1 by an initial (e.g., motion compensated) position shown in solid lines at θ₀ and the turned head position at the indicated angle θ shown by dash-dot lines. From the angle measurement θ (and optionally also from the images), the processor 30 is programmed to compute shifts of voxels of the image of the head resting in the head cradle 14 respective to reference positions of the voxels defined by the reference pivot angle θ₀ of the head cradle. As different voxels in general have different shifts depending upon how far away they are from the pivot axis A, the shifts are calculated respective to motion compensated positions of the voxels of the head resting in the head cradle 14 defined by the reference pivot angle θ₀ of the head cradle 14.

Advantageously, as will be shown elsewhere herein, the electronic processor 30 is programmed to compute the shifts of the voxels without using information about a size or shape of the head resting in the head cradle 14. In other words, the geometric formula for computing the shift of a given voxel from the measured angle θ is independent of the size of the head H, and is independent of the shape of the head H.

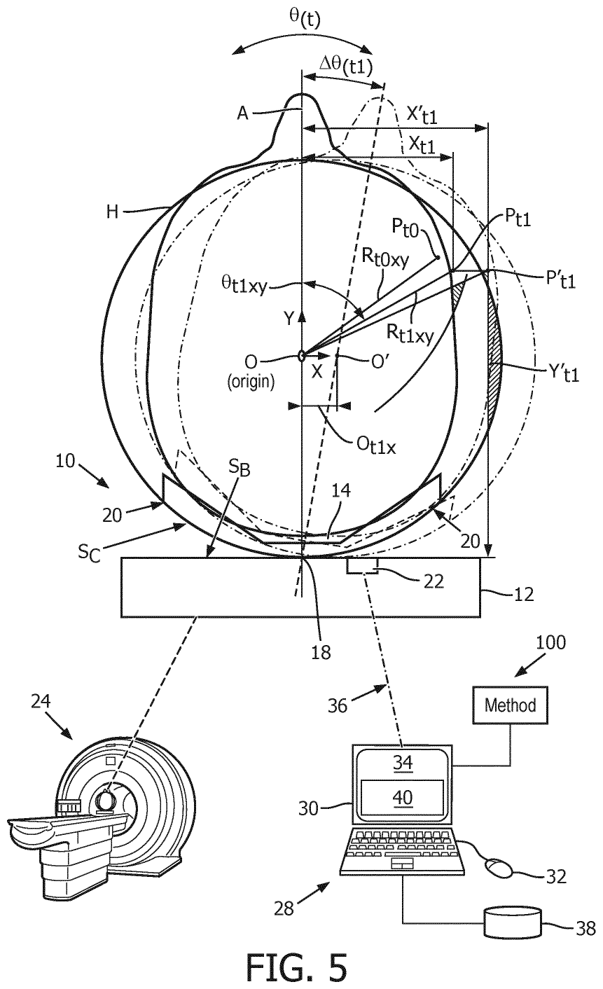

FIG. 2 shows another embodiment of a headrest 10′. The headrest 10′ is configured substantially identically to the head rest 10 of FIG. 1, except as described below. Instead of the pivot connection 16 shown in FIG. 1, the headrest 10′ of FIG. 2 includes a rolling connection 18 of the head cradle 14 with the base 12. Instead of measuring the pivot angle θ of the head cradle 14, the sensor 22 in the embodiment of FIG. 2 is configured to measure a roll position P (see FIG. 5) of the head cradle 14 over a surface S_{B} of the base 12. Specifically, the head cradle 14 is modified versus the embodiment of FIG. 1 by omitting the pivot connection 16 in favor of a rolling surface S_{C} of the cradle 14 that is supported by, and can roll across, the supporting surface S_{B} of the base 12. In the illustrative embodiment, the supporting surface S_{B} of the base 12 is flat, while the contacting surface S_{C} of the cradle 14 is a curved surface of constant radius, which facilitates computing the motion shift of voxels of the head H as a function of roll position P (described in further detail elsewhere herein with reference to FIG. 5; the roll position P has both rotation and translation components). The illustrative arrangement of FIG. 2 provides for roll in the side-to-side direction, analogous to the arrangement of FIG. 1. To achieve this, the surface S_{C} of the cradle 14 has a constant radius R_{C} with respect to an origin axis O running along the intersection of the sagittal and coronal planes, as indicated in FIG. 2. In this case, the rolling surface S_{C} is in the form of a cylindrical surface centered on the origin axis O. In other embodiments, the rolling surface S_{C} can also include a non-constant radius (not shown) with respect to the origin axis O. This non-constant radius can advantageously create a different feel during rolling of the patient's head within the head cradle 14. This configuration allows the patient to have a feel of being centered within the head cradle 14. The shape of the head cradle 14 provides a limited range of tilt, which maintains the head of the patient in a given tilt range. This will, however, require changes to compensation calculation. In one embodiment, if the rolling surface S_{C} is, for example elliptical, then an accurate correction for a linear portion of the coordinates would be different in regards to both the x-direction and a (small) y-direction component.

In some embodiments (not shown), the sensor 22 is also configured to measure roll position due to nodding motion of the head of the patient in a sagittal plane. To achieve this, the rolling surface S_{C} of the cradle 14 has the constant radius R_{C} with respect to the origin O which is now a voxel. In this case, the rolling surface S_{C} is in the form of a spherical surface centered on the origin voxel O, and a second sensor (not shown) measures the roll position due to nodding motion of the head.

As will be described elsewhere in more detail with reference to FIG. 5, the roll position of the cradle 14 of FIG. 2 in the side-to-side direction is defined as having both a translational component R relative to the origin O, and a rotational component θ relative to the origin O.

As with the embodiment of FIG. 1, an MR head coil 26 may be integrated with the base 12 (as diagrammatically shown) and/or with the head cradle 14.

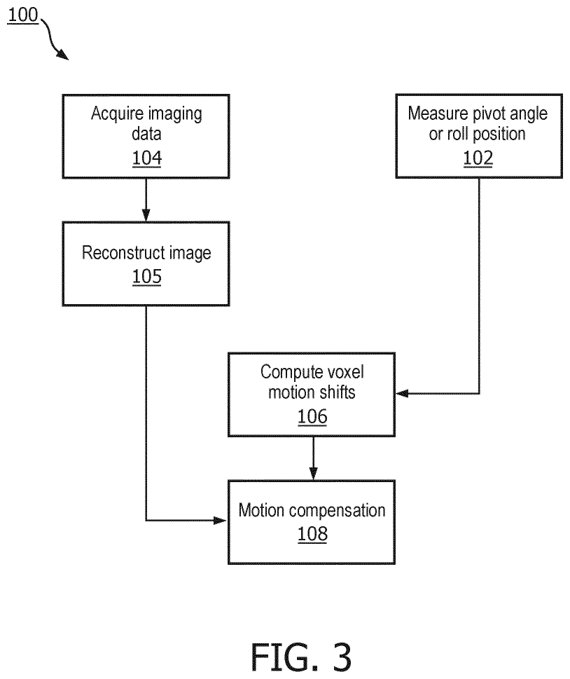

With reference to FIG. 3, an illustrative embodiment of a method 100 of measuring a motion shift of a head resting in a head cradle 14 having a pivot connection 16 or rolling connection 18 with a base 12. At 102, a pivot angle θ of the head cradle 14 about the pivot axis A of the pivot connection 16 of the head cradle (embodiment of FIG. 1) or the roll position P of the rolling connection 18 of the head cradle with the base (embodiment of FIG. 2) is measured with the sensor 22. At 104, the at least one electronic processor 30 is programmed to control the imaging device 24 to acquire imaging data of the head resting in the head cradle 14. The operations 102, 104 are preferably performed concurrently, that is, the magnetic resonance imaging data are acquired by the MRI scanner 24 in operation 104 and during this imaging data acquisition the pivot angle measurement 102 is performed. At 105, the at least one electronic processor 30 is programmed to reconstruct the imaging data acquired at 104 to form an image of the head H. The reconstruction algorithm employed at 105 suitably depends on the imaging modality of the acquisition 104 and other design choices. For example, in MRI imaging it is common to acquire k-space data at 104 and to employ a Fourier reconstruction at 105 to reconstruct the k-space data into an MRI image, although other MRI image reconstruction algorithms are contemplated depending on the spatial encoding used in the acquisition at 104. In the case of PET imaging, the reconstruction 105 may employ an iterative image reconstruction algorithm. These are merely examples. At 106, the at least one electronic processor 30 is programmed to compute motion shifts of voxels of the image of the head resting in the head cradle due to motion of the head using the measured pivot angle or roll position. These motion shifts are computed at 106 using only the pivot angle or roll position, by way of a geometric transform described elsewhere herein. At 108, the at least one electronic processor 30 is programmed to perform motion compensation on the image reconstructed at 105 using the voxel motion shifts computed at 106. In some embodiments, the motion compensation at 108 is performed using only the voxel motion shifts computed at 106. In other embodiments, the motion compensation at 108 is performed using the voxel motion shifts computed at 106 along with image information. For example, the voxel shifts computed at 106 by the geometric transform using the pivot angle or roll position may provide an initial motion compensated image; after which the motion compensation is further refined by comparison with an earlier image of the head H acquired with the cradle 14 at the reference pivot angle θ₀ (embodiment of FIG. 1) or at the reference roll position (embodiment of FIG. 2).

In the following, some examples of the voxel motion shift computation at 106 of FIG. 3 are described.

### Example 1 Calculation of Coordinates for the Headrest 10 with the Pivot Connection 16

Referring back to the headrest 10 of FIG. 1, the computing operation by the processor 30 includes determining motion corrected position of a voxel in the head resting in the head cradle 14 from a measured position of the voxel in the head resting in the head cradle. To do so, the processor 30 is programmed to determine a representative location a voxel of the head resting in the head cradle 14 at a first preselected moment from a measured position and at a second different preselected moment of the voxel of the head resting in the head cradle. The change is computed as a function of a change in the pivot angle measured by the sensor 22 as the voxel moves from the motion compensated position to the measured position and a distance of the voxel from a pivot axis of the pivot connection 16. The coordinates are calculated as: (Pₜ₁→Pₜ₀) Polar coordinate translation is (Rₜ₁, θₜ₂→−Δθ(t)). Pₜ₀ is the motion corrected position of a voxel in the head resting in the head cradle 14, Pₜ₁ is the measured position of the same voxel in the head resting in the head cradle at moment t1. R_{t1xy} is the distance of the measured voxel of interest from the pivot axis at moment t1. θₜ₀ is an initial reference pivot angle measured by the sensor 22 and θₜ₁ is a reference pivot angle measured by the sensor 22 at moment t1. Δθ(t) is change in the pivot angle as a function of time t.

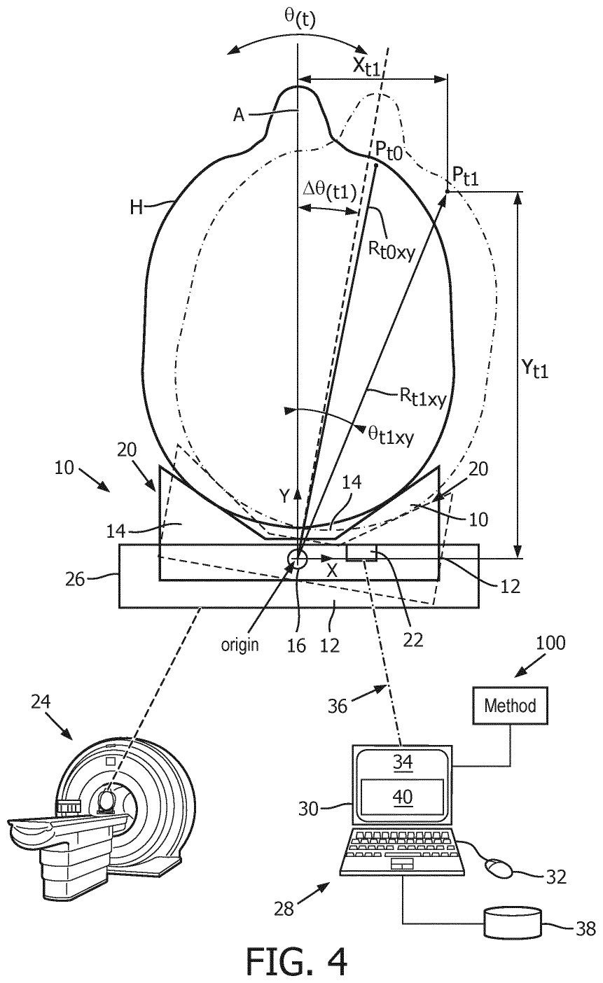

FIG. 4 shows an example of how the coordinates of head motion are calculated for the headrest 10. Cartesian coordinates (X, Y) are calculated and used to determine Polar coordinates (R, θ(t)). Both the Cartesian coordinates and the Polar coordinates are measured from an origin at a tilt axis (e.g., the pivot connection 16). The (X, Y coordinates) are measured at position Pₜ₁ on a patient's head (Pₜ₁ can be selected arbitrarily). The coordinates are determined along horizontal X-axis extending through the base 12 and a vertical Y-axis (e.g., along the centerline of a MM patient bore (not shown)), both of which intersect at the voxel connection 16. From these Cartesian coordinates (Xₜ₁, Yₜ₁) of the Pₜ₁ the Polar coordinate R_{t1xy} can be determined according to Equation 1:

R_{tlxy}=\sqrt{Xₜ₁²+Yₜ₁²}=R_{t0xy} (1)

Where R_{t0xy} is a Polar Coordinate R at the initial time to and R_{t1xy} is the Polar coordinate R at the time t1. Using Equation 1, R can be determined for all voxels independent of the time t.

The second Polar coordinate θ(t) can be continuously measured. An initial reference value θₜ₀ is measured at the initial time t0. Another reference angle measurement θₜ₁ is measured at the time t1. A change in the angle Δθ(t) can be determined by

Δθ(t)=θₜ₁−θₜ₀.

For an arbitrary Po the Polar coordinate θ(t) in described coordinate system can be determined according to Equation 2:

$${θ}_{t 1 xy} = {sin}^{- 1} ( \frac{{X}_{t 1}}{{Y}_{t 1}} ) \qquad ( 2 )$$

where θ_{t1xy} is the angle of the R_{t1xy} relative Y-axis. From this, θ_{t0xy} for any voxel can be calculated at the initial time t₀ according to Equation (3):

θ_{t0xy}=θ_{t1xy}−Δθ(t₁) (3)

Using these Polar coordinates (R, θ(t)), every image voxel can be compensated to its anatomical representative Pₜ₀ position at t=0 from a rotated Pₜ₁ location at (t=t₁) according to Equation 4:

Pₜ₁→Pₜ₀=(R,θ_{t1xy}−Δθ(t₁)) (4)

### Example 2 Calculation of Coordinates for the Headrest 10′ with the Rolling Connection

Referring back to the headrest 10′ of FIG. 2, the processor 30 is configured to compute shifts of voxels of the image of the head resting in the head cradle respective to reference positions of the voxels defined by a reference roll angle θₜ₀ of the head cradle from a roll angle measurement θ(t) received from the sensor 22.

FIG. 5 shows an example of how the coordinates of head motion are calculated for the headrest 10′. In this embodiment, there is a rotation due the sideways tilting of the head (similar to that described in EXAMPLE 1), along with linear motion due the rolling along the surface of the head cradle 14. For transferring the position of the voxel of interest to t=t₀ from position at t=t₁, linear motion of the origin O to a new, measured position O′ due to the rolling being calculated first. A measured position P′ₜ₁ can now be compensated for a linear part of the motion and returned to position Pₜ₁. After this, the voxel Pₜ₁ can be compensated for rotational part and returned to position Pₜ₀ according to P′ₜ₁→Pₜ₁→Pₜ₀. The origin O in this embodiment is the rolling radius center of the contacting surface S_{C}.

Similar to EXAMPLE 1, a change in the angle Δθ(t₁) can be determined according to Δθ(t₁)=θₜ₁−θₜ₀. From this, a distance traveled by the origin (O→O′) in the X-direction (ΔO_{t1X}) can be determined by Equation 5:

ΔO_{t1X}=Δθ(t₁)*R_{C} (5)

Using ΔO_{t1X}, the linear motion compensated coordinates at time t₁ (Xₜ₁, Yₜ₁) can be determined according to Equations (6) and (7):

Xₜ₁=X′ₜ₁−ΔO_{t1X} (6)

Yₜ₁=Y′ₜ₁ (7)

where X′ₜ₁ and Y′ₜ₁ are Cartesian coordinates acquired at the time t₁; and Xₜ₁ and Yₜ₁ are linear motion compensated coordinates to be used in the rotational compensation at time t₁. Using the Cartesian coordinates X′ₜ₁ and Y′ₜ₁, every image voxel can be compensated for linear portion of rolling motion to position Pₜ₁ from a linearly shifted position P′ₜ₁ according to Equation (8):

P′ₜ₁→Pₜ₁=(X′ₜ₁−ΔO_{t1X},Y′ₜ₁), (8)

Now by using calculated Xₜ₁ and Yₜ₁ and equations 2, 3 and 4 from EXAMPLE 1, the motion compensation can be completed.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A headrest for an imaging device, the headrest comprising:
a base;
a head cradle having a pivot connection or rolling connection with the base; and
a sensor configured to measure a pivot angle (θ) of the head cradle about a pivot axis (A) of the pivot connection of the head cradle with the base or a roll position (P) of the rolling connection of the head cradle with the base.

2. The headrest of claim 1, wherein the head cradle has a pivot connection with the base and the sensor is configured to measure the pivot angle (θ) of the head cradle about the pivot axis of the pivot connection of the head cradle with the base.

3. The headrest of claim 2, further including:
at least one electronic processor operatively connected with the sensor, the at least one electronic processor being programmed to:
receive a pivot angle measurement of the pivot angle (θ) of the head cradle from the sensor;
receive an image of a head resting in the head cradle from the imaging device; and
compute shifts of voxels of the image of the head resting in the head cradle respective to reference positions of the voxels defined by a reference pivot angle (θ₀) of the head cradle from the received pivot angle measurement.

4. The headrest of claim 3, wherein the computing comprises:
determining a representative location a voxel of the head resting in the head cradle at a first preselected moment from a measured position and at a second different preselected moment of the voxel of the head resting in the head cradle, the change being computed as a function of a change in the pivot angle measured by the sensor as the voxel moves and a distance of the voxel from a pivot axis of the pivot connection.

5. The headrest of claim 4, wherein the coordinates are calculated as:
$${P}_{t 1} \to {P}_{t 0} , ( R , {θ}_{t 1} - Δθ ( t ) )$$
wherein Pₜ₀ is the motion compensated position of a voxel in the head resting in the head cradle, Pₜ₁ is the measured position of the voxel in the head resting in the head cradle, R is the distance of the voxel from the pivot axis, θₜ₀ is a reference pivot angle measured by the sensor, and Δθ(t) is change in the pivot angle compared to θₜ₀ measured by the sensor as a function of time t.

6. The headrest of claim 1, wherein the head cradle has a rolling connection with the base and the sensors is configured to measure the roll position of the rolling connection of the head cradle with the base.

7. The headrest of claim 6, further including:
at least one electronic processor operatively connected with the sensor, the at least one electronic processor being programmed to:
receive a roll position measurement of the roll position of the head cradle from the sensor;
receive an image of a head resting in the head cradle from the imaging device; and
compute shifts of voxels of the image of the head resting in the head cradle; and
compensate for positions of the voxels based on a measured roll position θ(t) of the head cradle from the received roll position measurement and measured coordinates of the voxels.

8. The headrest of claim 7, wherein the computing comprises:
determining a motion compensated position of a voxel in the head resting in the head cradle to a measured position of the voxel in the head resting in the head cradle, the change being computed as a function of a change in the pivot angle measured by the sensor as the voxel moves from the motion compensated position to the measured position and a distance of the voxel from the pivot axis.

9. The headrest of claim 8, wherein the coordinates are calculated as:
$${p}_{t 1}^{′} \to {p}_{t 1} \to {P}_{t 0} {X}_{t 1 xy} = {X}_{t 1 xy}^{′} - Δθ ( t ) * {R}_{C} and {Y}_{t 1 xy} = {Y}_{t 1 xy}^{′} , \\ {R}_{t 1 xy} = \sqrt{{X}_{t 1}^{2} + {Y}_{t 1}^{2}} \to ( {R}_{t 1 xy} , {θ}_{t 1} - Δθ ( t ) )$$
wherein Pₜ₀ is the motion compensated position of a voxel in the head resting in the head cradle, P′ₜ₁ is the measured position of the voxel in the head resting in the head cradle, Pₜ₁ is the linear motion component compensated position of the voxel in the head resting in the head cradle, R_{t1xy} is the distance of the voxel Pₜ₁ from the Origin (O), θₜ₀ is an initial reference angle measured by the sensor at t=t0, Δθ(t) is the change in the pivot angle compared to θₜ₀ measured by the sensor as a function of time t and R_{C} is a radius for a roll surface of the head in the head cradle.

10. The headrest of claim 6, wherein the sensor is configured to measure roll position due to nodding motion of the head of the patient in a sagittal plane.

11. The headrest of claim 1, wherein the sensor is configured to measure the pivot angle or roll position for side-to-side rotational motion of the head of the patient in an axial plane.

12. The headrest of claim 1, wherein the head cradle includes wedge-shaped portions disposed at opposing ends of the head cradle to receive a head of a patient to be imaged.

13. The headrest of claim 1, wherein the imaging device is a magnetic resonance (MR) imaging device and the headrest further comprises:
an MR head coil disposed in or on the head cradle and/or the base.

14. The headrest of claim 1, further including:
at least one electronic processor operatively connected with the sensor, the at least one electronic processor being programmed to compute shifts of voxels of an image of a head resting in the head cradle respective to a reference position of the head defined by a reference pivot angle or roll position of the head cradle using the pivot angle or roll position measured by the sensor;
wherein the at least one electronic processor is programmed to compute the shifts of the voxels without using information about a size or shape of the head resting in the head cradle.

15. The headrest of claim 1, wherein the sensor includes an inclinometer, a laser-based optical sensor, or a rotational encoder.

16. The headrest of claim 1, further comprising,
an imaging device configured to obtain one or more images of the head of the patient disposed in the cradle, the imaging device being one of a Magnetic Resonance imaging device or a Computed Tomography imaging device.

17. A method of measuring a motion shift of a head resting in a head cradle having a pivot connection or rolling connection with a base, the method comprising:
using a sensor, measuring a pivot angle of the head cradle about a pivot axis (A) of the pivot connection of the head cradle with the base or a roll position of the rolling connection of the head cradle with the base;
using an imaging device, acquiring an image of the head resting in the head cradle; and
with at least one electronic processor, computing motion shifts of voxels of the image of the head resting in the head cradle due to motion of the head using the measured pivot angle or roll position.

18. The method of claim 17, further including, with the at least one electronic processor:
receiving a pivot angle measurement of the pivot angle of the head cradle from the sensor;
receiving an image of a head resting in the head cradle from the imaging device; and
computing shifts of voxels of the image of the head resting in the head cradle respective to reference positions of the voxels defined by a reference pivot angle (θ₀) of the head cradle from the received pivot angle measurement.

19. The method of claim 17, further including, with the at least one electronic processor:
receiving a roll position measurement of the roll position (P) of the head cradle from the sensor;
receiving an image of a head resting in the head cradle from the imaging device;
computing shifts of voxels of the image of the head resting in the head cradle; and
compensating for positions of the voxels based on a measured roll position θ(t) of the head cradle from the received roll position measurement and measured coordinates of the voxels.

20. The method of claim 17, further including, with the at least one electronic processor:
computing shifts of voxels of an image of a head resting in the head cradle respective to a reference position of the head defined by a reference pivot angle or roll position of the head cradle using the pivot angle or roll position measured by the sensor, the computing including computing the shifts of the voxels without using information about a size or shape of the head resting in the head cradle.
